(19) United States
(12) Patent Application Publication
(10) Pub. No.: US 2026/0124063 A1
(43) Pub. Date: May 7, 2026

(54) DEVICES TO TREAT AND/OR ENHANCE NAIL GROWTH

(71) Applicant: Christopher B. STEWART, Charlottesville, VA (US)

(72) Inventor: Christopher B. STEWART, Charlottesville, VA (US)

(21) Appl. No.: 19/349,701

(22) Filed: Oct. 3, 2025

Related U.S. Application Data
(60) Provisional application No. 63/715,697, filed on Nov. 4, 2024.

(51) Int. Cl. A61F 5/11

(52) U.S. Cl. CPC A61F 5/11

(57) ABSTRACT

Devices to assist in treatment and/or enhancement of nail growth of a digit for a patient in need thereof are provided with an anchor member adapted to being affixed to a subjacent portion of the nail bed of a patient's digit, an anchor strip and a traction strip. The anchor strip is adapted to being wrapped around the patient's digit proximally of the nail bed. The traction strip is adapted to being connected to the affixed anchor member so as to extend anteriorly around a distal end of the patient's digit and redirected posteriorly under the digit to allow an opposite end thereof to be attached to the anchor strip. The traction strip is thereby capable of exerting a predetermined tension to the nail bed of the patient to assist in treatment and/or enhancement of nail growth by attachment thereof to the anchor member affixed to the nail bed.

### CROSS REFERENCE TO RELATED APPLICATIONS

This application is based on and claims domestic priority benefits from U.S. Provisional Application Ser. No. 63/715,697 filed on Nov. 4, 2024, the entire content of which is expressly incorporated hereinto by reference.

### FIELD

The embodiments disclosed herein relate generally to devices that are especially useful to promote nail health and growth, particularly following trauma or surgical removal of the nail. Specifically, the embodiments of the devices disclosed herein offer a relatively gentle distraction designed to encourage physiological processes that enhance nail regeneration and directional growth.

### BACKGROUND

Following traumatic or surgical nail removal, patients often experience delayed and/or angulated nail growth and discomfort. This period of growth can range from months to greater than one year in some instances. Traditional methods for managing pain and anxiety during recovery include medications and topical treatments, but these do not specifically address the need for physiologic distraction and the encouragement of nail regrowth. Human nails are protective by design. It is well understood that the sooner nails grow back the sooner they can perform their protective function thereby allowing individuals to resume normal activity without pain or hesitation.

Various nail corrective devices are known in the art, e.g., from U.S. Pat. Nos. 8,137,297 and 8,262,600, the entire contents of each being expressly incorporated hereinto by reference. While such conventional nail corrective devices may serve their intended purposes, improvements are still desired. It is therefore towards providing such improvements that the embodiments of the present invention disclosed herein are directed.

### SUMMARY

Broadly, the embodiments disclosed herein are directed toward devices that are especially useful to promote nail health and growth by exerting a directional tension force onto a patient's nail. The direction tension force may by symmetrical to encourage nail growth in a symmetrical direction coincident to a patient's digit midline or may be angularly asymmetrical to redirect nail growth which is angularly offset relative to the patient's digit midline. The devices disclosed herein may be employed for nail growth enhancement for fingers and/or toes of a patient. The devices of the embodiments disclosed herein are thereby usefully employed so as to, for example, increase nail growth, correct angular deformities of nails and/or treat ingrown nails by redirecting the direction of nail growth.

According to exemplary embodiments, devices to assist in treatment and/or enhancement of nail growth of a digit for a patient in need thereof are provided with an anchor member adapted to being affixed to a portion of a nail of the patient's digit. An anchor strip having a sufficient length is adapted to be wrapped around the digit proximally of the nail and is connectable to one end of a traction strip. The traction strip is of sufficient length so as to be extended anteriorly around a distal end of the patient's digit and redirected posteriorly under the digit to allow an opposite end thereof to be attached to the anchor strip. The traction strip is thereby capable of exerting a predetermined tension to the nail of the patient to assist in treatment and/or enhancement of nail growth by attachment thereof to the anchor member affixed to the nail.

An adhesive material may be provided which is adapted to adhesively affix the anchor member to the nail of the patient. At least one (preferably both) of the anchor strip and the traction strip may be a conventional flexible hook-and-loop fastener strip (e.g., VELCRO-Brand fastener) having hooks on one surface and loops on an opposite surface thereof.

According to some embodiments, the anchor member will include a base element adapted to being adhesively affixed to the nail of the patient and a J-shaped anchor element which is oriented posteriorly when the anchor member is affixed to the nail of the patient. Alternatively, the anchor member the anchor member is in the form of a substantially flat tab having one end adapted to being adhesively affixed to the nail of the patient and an opposite unaffixed free end. The opposite end of the flat anchor tab member may define an opening so as to allow connection to one end of the traction strip.

In use, the anchor member is affixed to a subjacent portion of the nail while the anchor strip is wrapped around a patient digit posteriorly of the nail. One end of the traction strip may thereby be connected to the anchor member such that the traction strip extends anteriorly around and over a distal end of the patient's digit and is redirected posteriorly under the patient's digit. An opposite end of the traction strip may then be connected to the anchor strip so that the traction strip exerts a predetermined tension force on the nail of the patient through the anchor member affixed thereto.

These and other aspects and advantages of the present invention will become clearer after careful consideration is given to the following detailed description of the preferred exemplary embodiments thereof.

### BRIEF DESCRIPTION OF ACCOMPANYING DRAWINGS

The disclosed embodiments of the present invention will be better and more completely understood by referring to the following detailed description of exemplary non-limiting illustrative embodiments in conjunction with the drawings of which:

FIG. 1 is a plan view of kit components for a device in accordance with an embodiment of the invention which is adapted to treat and/or enhance nail growth of a patient in need of the same;

FIGS. 2-7 show an exemplary sequence of assembling the kit components shown in FIG. 1 onto a nail of a patient to construct the device in accordance with the invention to treat and/or enhance the patient's nail growth;

FIG. 8 is an enlarged plan view showing an alternative embodiment of an anchor member that may be employed in the kit of FIG. 1; and

FIG. 9 is a perspective view showing the manner in which the anchor member of FIG. 8 can be installed during the assembly of the device in accordance with another embodiment of the invention.

### DETAILED DESCRIPTION OF EXEMPLARY EMBODIMENT

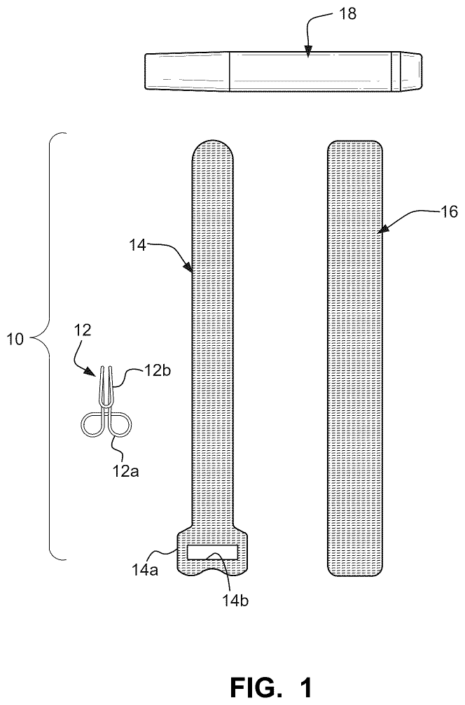

Accompanying FIG. 1 depicts the component parts of a device in kit form 10 which are adapted to being assembled to form a device that assists in the treatment and/or enhancement of nail growth for a patient in need thereof. In essence, the device kit 10 will include an anchor member 12, an elongate traction strip 14 and an elongate anchor strip 16.

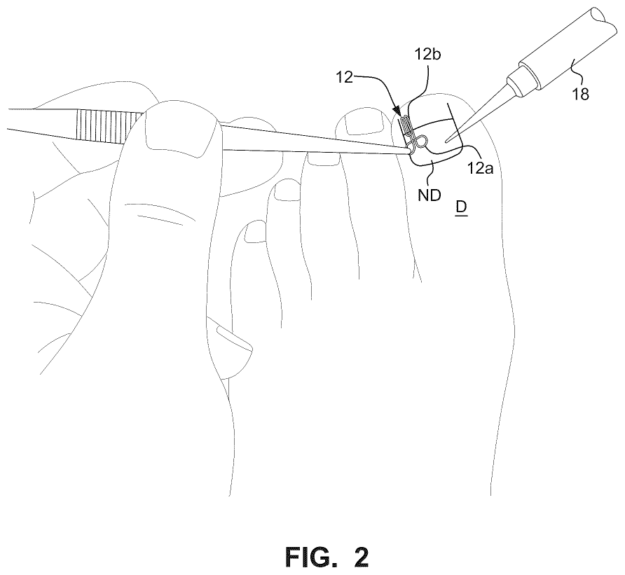

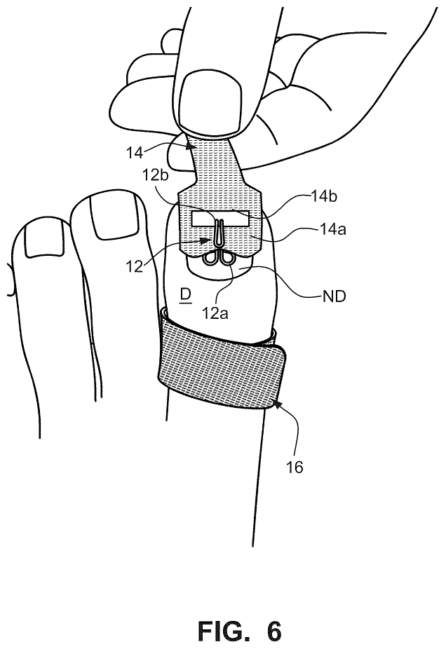

In the embodiment shown in FIGS. 1-7, the anchor member 12 is in the form of a J-shaped component having a base portion 12a and a hook portion 12b. The base portion 12a is adapted to being immovably fixed to a portion of the patient's nail NP (see FIGS. 2-7) by a suitable nail compatible adhesive. In this regard, the device kit 10 may include a vial 18 containing a suitable adhesive, e.g., a liquid cyanoacrylate adhesive that is conventionally employed to attach synthetic nails to natural nails. When cured, therefore, the adhesive will immovably fix the base portion 12a of the anchor member 12 to a desired subjacent portion of the patient's nail NP such that the hook portion 12b extends upwardly and posteriorly relative to the nail NP. The anchor member 12 can be formed of any metal or plastic material that is compatible with the adhesive. In the embodiment depicted, the anchor member is in the form of a metal wire element that is bent into a J-shaped member forming the base and hook members 12a and 12b, respectively.

Each of the elongate traction and anchor strips 14 and 16, respectively, are preferably flexible strips of hook-and-loop fasteners (e.g., VELCRO-Brand fasteners) having a dense plurality of hooks on one side surface thereof and a dense plurality of pile loops on an opposite side surface. As is well known, a secure yet releasable attachment occurs when a portion of the hooks on one side of the fastener are brought into engagement with a corresponding portion of the loops on the opposite side of the fastener.

The traction strip 14 may include a head portion 14a at one end thereof which defines a transverse slot 14b. The traction strip 14 may thus be in the form of a conventional flexible tie wrap, such as a VELCRO-Brand ONE WRAP® Tie element. The anchor strip 16 can be the same or different from the traction strip 14. Thus, in the embodiment shown, the anchor strip 16 is in the form of an elongate VELCRO-Brand strip that does not include a head portion. However, VELCRO-Brand ONE WRAP® Tie element similar to that employed for the traction strip 14 could also suitably be employed as the anchor strip 16.

An exemplary sequence of component assembly of the device kit 10 is shown in FIGS. 2-7. In this regard, the anchor member 12 is initially attached to a desired surface portion of the patient's nail NP associated with a patient's digit D1 (which in the embodiment shown is exemplified by the patient's large toe but could be any other digit of the patient) by applying a suitable amount of adhesive from the vial 18 and then positioning the base element 12a of the anchor member 12 onto the applied adhesive such that the J-shaped hook element 12b is oriented in a posterior direction. Once the adhesive has cured, the anchor member 12 will thus be immovably fixed to the nail NP.

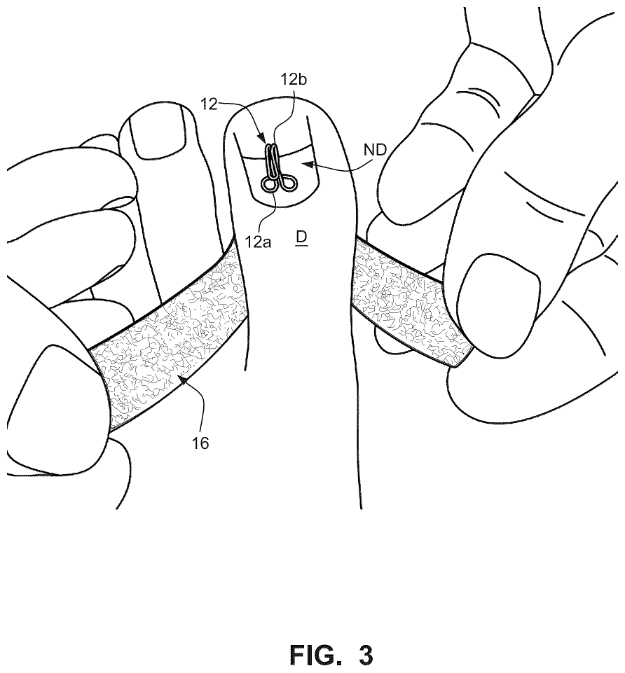

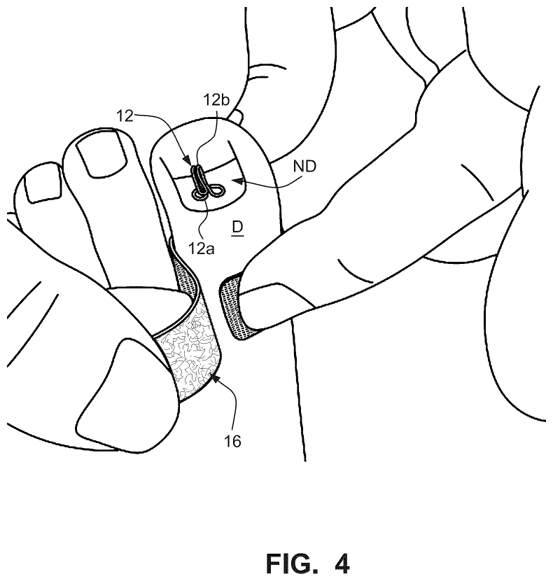

During or after curing of the adhesive which fixes the anchor member 12, the anchor strip 16 may be wrapped around the patient's digit D proximally (posteriorly) of the nail NP as shown in FIGS. 3-4 and secured to itself as shown in FIG. 4 by bringing the loop side of the strip 16 into engagement with respective portions of the hook side of the strip 16. When wrapped around the patient's digit D, the loop side of the strip 16 is preferably the bottom side so as to contact the patient's skin and thereby promote comfort, while the hook side is the upper side of the strip 16 to thereby engage a portion of the loop side after the strip 16 is wrapped around the digit D.

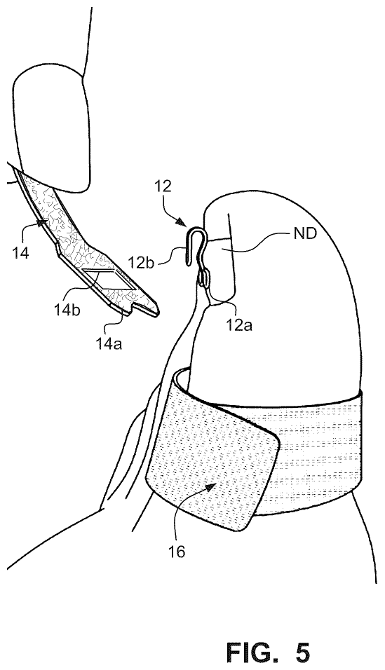

As is shown in FIGS. 5-7, the traction strip 14 may then be positioned relative to the distal (anterior) tip end of the patient's digit D with the loop side preferably facing down so it is against the patient's skin. The slot 14b defined by the head 14a of the traction strip 14 will receive the J-shaped hook member 12b to allow the traction strip 14 to be extended around the distal (anterior) tip end of the patient's digit D and then pulled proximally (posteriorly) under the digit D to allow an end portion of the traction strip 14 to be engaged with a corresponding portion of the anchor strip (e.g., by bringing the loops of the end portion of the traction strip 14 into engagement with a corresponding portion of the hooks of the anchor strip 16). It will be appreciated that once secured to the anchor strip 16, the traction strip 14 will exert a desired amount of tension force which is transferred onto the nail NP by the anchor member 12.

The tension force applied to the nail NP by the traction strip 14 when installed in the manner described above may be symmetrical (e.g., the tension force will be exerted on the nail NP in substantial alignment with the centerline of the digit D) or asymmetrical (e.g., the tension force will be offset relative to the centerline of the digit D).

Symmetrical tension force is desired if the nail NP is itself substantially symmetrical and non-angulated. In those situations where the nail NP is asymmetrically angulated, it may be desirable to exert a greater tension force on one side of the nail NP as compared to the other.

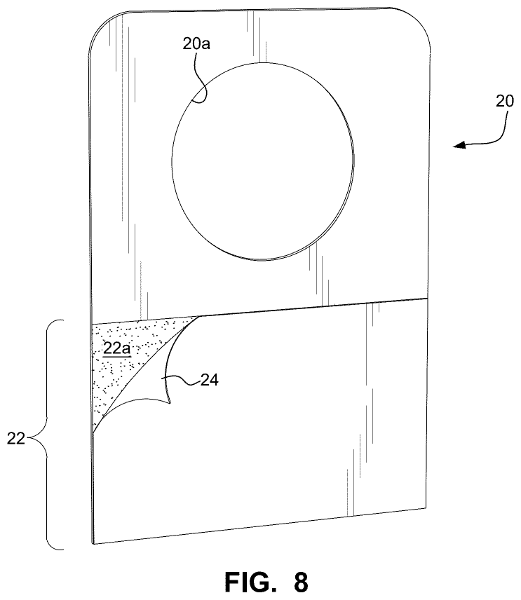

An alternative embodiment of the anchor member 20 to the anchor member 12 discussed previously is shown in FIG. 8. Specifically, the anchor member 20 as shown in the embodiment of FIG. 8 is preferably a flat sheet-like tab formed from plastic or metal material. A lower underside portion of the anchor member 20 may be provided with an adhesive region 22 is provided with a layer of adhesive 22a covered by a protective release film 24 prior to use. The anterior end portion of the anchor member 20 defines an opening 20a that is sized and configured to receive therein the traction strip 14 as described previously.

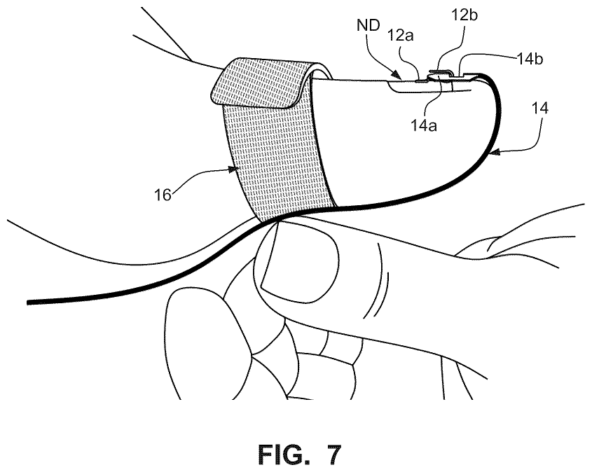

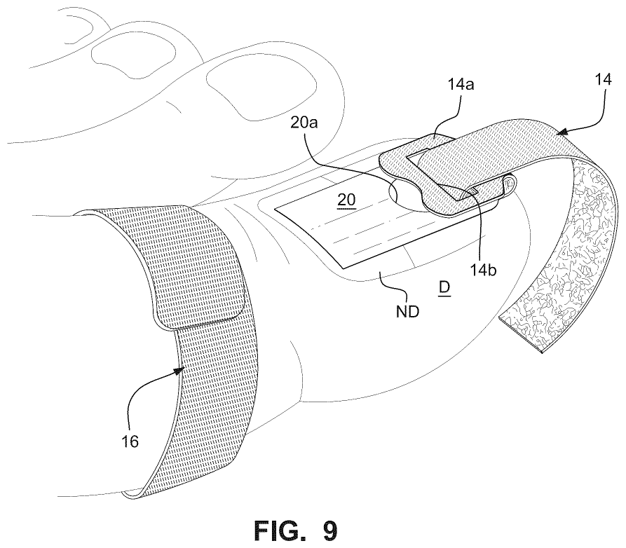

In order to assemble the anchor member 20 to the nail NP, the release film 24 is first removed to expose the adhesive region 22. The adhesive region 22 may thus be brought into contact with a surface portion of the nail NP so that anterior portion of the anchor member which defines the opening 20a is positioned near the distal end of the patient's digit D. The anterior portion of the anchor member 20 is therefore unattached to the nail NP and may be flexed upwardly sufficiently to allow the traction strip 14 to be looped through the opening 20a. Such a state is shown in FIG. 9. Alternatively, the traction strip 14 may be looped through the opening 20a prior to securing the anchor member 20 to the nail NP. Thereafter, the traction strip 14 may be secured to the anchor strip 16 in a manner similar to that shown in FIG. 7 and described hereinabove.

### Example

A patient who had two slow growing nails (left and right great toenails) applied the device as shown and described above to only the slow growing left great toenail to determine if the device affected growth of that nail and whether there was a difference in growth with the other slow growing right great toenail, that was thereby used as a “control”. After a 3 month period of time, the before and after pictures of the patient's left and right great toenails showed a 50-60% increase in nail growth of the left great toenail to which the device was applied as compared to the control right great toenail.

While reference is made herein to particular embodiments of the invention, various modifications within the skill of those in the art may be envisioned. Therefore, it is to be understood that the invention is not to be limited to the disclosed embodiment, but on the contrary, is intended to cover various modifications and equivalent arrangements included within the spirit and scope thereof.

1. A device to assist in treatment and/or enhancement of nail growth of a digit for a patient in need thereof, the device comprising:
an anchor member adapted to being affixed to a subjacent portion of a nail of the digit;
an anchor strip having a sufficient length to be wrapped around the digit proximally of the nail; and
a flexible traction strip having one end connectable to the anchor member and being of sufficient length so as to be adapted for extension anteriorly around a distal end of the digit and redirected posteriorly under the digit to allow an opposite end of the traction strip to be attached to the anchor strip, wherein
the flexible traction strip is capable of exerting a predetermined tension to the nail of the patient to assist in treatment and/or enhancement of nail growth by attachment thereof to the anchor member affixed to the nail.

2. The device according to claim 1, further comprising an adhesive material which is adapted to adhesively affix the anchor member to the nail of the patient.

3. The device according to claim 1, wherein at least one of the anchor strip and the traction strip is a hook-and-loop fastener strip having hooks on one surface and loops on an opposite surface.

4. The device according to claim 1, wherein each of the anchor strip and the traction strip is a hook-and-loop fastener strip having hooks on one surface and loops on an opposite surface.

5. The device according to claim 1, wherein the anchor member includes a base element adapted to being affixed to the nail of the patient and a J-shaped anchor element which is oriented posteriorly when the anchor member is affixed to the nail of the patient.

6. The device according to claim 4, further comprising an adhesive material which is adapted to adhesively affix the base element of the anchor member to the nail of the patient.

7. The device according to claim 1, wherein the anchor member is a substantially flat tab having one end adapted to being affixed to the nail of the patient and an opposite unaffixed free end.

8. The device according to claim 7, wherein the opposite unaffixed free end of the anchor member defines an opening adapted to being connected to the flexible traction strip.

9. The device according to claim 8, wherein the one end of the anchor member includes an adhesive region which is adapted to adhesively affix the one end of the anchor member to a subjacent portion of the nail of the patient.

10. The device according to claim 9, wherein anchor member further includes a removable protective release film covering the adhesive region.

11. A method to treat and/or enhance nail growth of a patient in need thereof, the method comprising:
(i) providing the device according to claim 1;
(ii) affixing the anchor member to a subjacent portion of the nail;
(iii) wrapping an anchor strip around the patient's digit posteriorly of the nail;
(iv) connecting the one end of the traction strip to the anchor member and extending the traction strip anteriorly around a distal end of the patient's digit and then redirecting the traction strip posteriorly under the patient's digit; and
(v) attaching an opposite end of the traction strip to the anchor strip so that the traction strip exerts a predetermined tension force on the nail of the patient through the anchor member affixed thereto.

12. The method according to claim 11, wherein each of the anchor strip and the traction strip is a hook-and-loop fastener strip having hooks on one surface and loops on an opposite surface.

13. The method according to claim 11, wherein the anchor member includes a base element adapted to being affixed to the nail of the patient and a J-shaped anchor element which is oriented posteriorly when the anchor member is affixed to the nail of the patient.

14. The method according to claim 11, wherein step (ii) includes affixing the anchor member to the nail with adhesive.

15. The method according to claim 14, wherein step (ii) includes applying a liquid adhesive to a portion of the nail, bringing the anchor member into contact with the applied liquid adhesive and allowing the adhesive to solidify.

16. The method according to claim 11, wherein the anchor member is a substantially flat tab having one end adapted to being affixed to the nail of the patient and an opposite unaffixed free end, and wherein step (ii) includes affixing the one end of the anchor member to the nail of the patient.

17. The method according to claim 16, wherein step (ii) includes providing a lower portion of the anchor member with an adhesive region and bringing the adhesive region of the anchor member into contact with the nail.

18. The method according to claim 17, wherein the anchor member includes a removable protective release film covering the adhesive region, and wherein step (ii) includes removing the protecting release film to expose the adhesive region, and thereafter bringing the adhesive region into contact with the nail.
